(19) United States
(12) Patent Application Publication
(10) Pub. No.: US 2025/0161513 A1
(43) Pub. Date: May 22, 2025

(54) DETERMINING AN APPLICATION CONDITION OF STERILANT TO ACHIEVE A STERILITY ASSURANCE LEVEL

(71) Applicant: Ethicon, Inc., Raritan, NJ (US)

(72) Inventors: Bradley Lundahl, Peoria, AZ (US); Jagdishchanr Patel, Lawrenceville, NJ (US)

(21) Appl. No.: 18/517,552

(22) Filed: Nov. 22, 2023

(51) Int. Cl. A61L 2/28

(52) U.S. Cl. CPC A61L 2/28

(57) ABSTRACT

A system may determine an application condition of sterilant to achieve a sterility assurance level (SAL) for an article. The system may determine a hypothesized sterilant resistance (Θ_{HS}) that indicates a probability of a micro-organism expressing growth once exposed to a unit of sterilant; determine, based, at least in part, on the hypothesized sterilant resistance, an estimated number of samples that include a micro-organism expressing growth once exposed to the unit of sterilant; determine an observed number of samples that include a micro-organism expressing growth subsequent to exposure to the unit of sterilant; iteratively determine, based on one or more iterations, at least one first correction based on the observed number and the estimated number, and apply convergence criteria to determine whether to perform a next iteration; and determine the application condition of the sterilant, based on the first correction. Other aspects are also described and claimed.

### FIELD

This disclosure relates generally to sterility assurance, and more specifically to determining an application condition of sterilant to achieve a sterility assurance level (SAL) for an article. Other embodiments are also described.

### BACKGROUND

An SAL refers to a probability that an article that has been exposed to a sterilant may nevertheless remain non-sterile, e.g., the article may continue to have a micro-organism expressing growth despite exposure to the sterilant. For example, achieving an SAL of 10⁻⁶ for an article may indicate a probability of one in one million that the article will continue to have a micro-organism expressing growth despite exposure to the sterilant.

In various applications, different sterilant modalities may be used and different SALs may be targeted. For example, sterilant modalities for an article could include one or more of radiation, Ethylene Oxide, dry heat, moist heat, and vaporized hydrogen peroxide. Further, an SAL may be targeted based on a device classifications for an article. For example, an article that may have a direct blood path, such as a syringe, may require a relatively higher SAL (e.g., 10⁻⁶). In another example, an article that does not have a direct blood path, such as a cup or bowl, may be acceptable at a relatively lower SAL (e.g., 10⁻¹).

### SUMMARY

A system may utilize one or more machine learning algorithms to learn a microbiological quality and quantify a sterilant resistance of a microbial population. This may enable determining an application condition of sterilant to achieve an SAL for an article. In implementations, the one or more machine learning algorithms may include an expectation-maximization (EM) algorithm and/or a Bayesian maximum a posteriori (MAP) algorithm. The algorithm, whether EM or Bayesian MAP, may be applied once or iteratively to estimate a value of a latent variable, e.g., Theta (Θ). In implementations, one of the algorithms can be used independently, e.g., the EM algorithm, or multiple algorithms can be combined, e.g., the EM algorithm and the Bayesian MAP algorithm used sequentially. The EM algorithm can generate an EM estimate which may be a maximum log likelihood estimate. The EM estimate may be analogous to a Bayesian MAP estimate. In implementations, the system can apply a single E-operation M-operation estimate, a multiple E-operation M-operation iteration estimate, a single operation Bayesian MAP estimate, a multiple operation Bayesian MAP estimate, or a combination thereof (e.g., a single operation Bayesian MAP estimate followed by a single E-operation M-operation estimate, a single E-operation M-operation estimate followed by a single operation Bayesian MAP estimate, or another sequence). As a result, the system can determine optimum application conditions of a sterilant to apply to an article to achieve an SAL. For example, the system can specify an amount, an absorbed dose, and/or a duration of exposure, including at a given temperature, and/or pressure, for a given sterilant modality to achieve a minimum SAL given a microbial resistance of an actual article.

Some implementations may include a method for determining an application condition of sterilant to achieve an SAL for an article. The method may include determining a hypothesized sterilant resistance (Θ_{HS}) that indicates a probability of a micro-organism expressing growth once exposed to a unit of sterilant; determining, based, at least in part, on the hypothesized sterilant resistance, an estimated number of samples that include a micro-organism expressing growth once exposed to the unit of sterilant; determining an observed number of samples that include a micro-organism expressing growth subsequent to exposure to the unit of sterilant; iteratively determining, based on one or more iterations, at least one first correction based on the observed number and the estimated number, and applying convergence criteria to determine whether to perform a next iteration; and determining the application condition of the sterilant, based on the first correction, and applying the application condition to the article to achieve the SAL.

Some implementations may include a non-transitory computer readable medium storing instructions operable to cause one or more processors to perform operations. The operations may include determining a hypothesized sterilant resistance (Θ_{HS}) that indicates a probability of a micro-organism expressing growth once exposed to a unit of sterilant; determining, based, at least in part, on the hypothesized sterilant resistance, an estimated number of samples that include a micro-organism expressing growth once exposed to the unit of sterilant; determining an observed number of samples that include a micro-organism expressing growth subsequent to exposure to the unit of sterilant; iteratively determining, based on one or more iterations, at least one first correction based on the observed number and the estimated number, and applying convergence criteria to determine whether to perform a next iteration; and determining the application condition of the sterilant, based on the first correction, and applying the application condition to the article to achieve the SAL.

Some implementations may include an apparatus comprising a memory and a processor configured to execute instructions stored in the memory to determine a hypothesized sterilant resistance (Θ_{HS}) that indicates a probability of a micro-organism expressing growth once exposed to a unit of sterilant; determine, based, at least in part, on the hypothesized sterilant resistance, an estimated number of samples that include a micro-organism expressing growth once exposed to the unit of sterilant; determine an observed number of samples that include a micro-organism expressing growth subsequent to exposure to the unit of sterilant; iteratively determine, based on one or more iterations, at least one first correction based on the observed number and the estimated number, and apply convergence criteria to determine whether to perform a next iteration; and determine the application condition of the sterilant, based on the first correction, and apply the application condition to the article to achieve the SAL.

The above summary does not include an exhaustive list of all aspects of the present disclosure. It is contemplated that the disclosure includes all systems and methods that can be practiced from all suitable combinations of the various aspects summarized above, as well as those disclosed in the Detailed Description below and particularly pointed out in the Claims section. Such combinations may have particular advantages not specifically recited in the above summary.

### BRIEF DESCRIPTION OF THE DRAWINGS

Several aspects of the disclosure here are illustrated by way of example and not by way of limitation in the figures of the accompanying drawings in which like references indicate similar elements. It should be noted that references to “an” or “one” aspect in this disclosure are not necessarily to the same aspect, and they mean at least one. Also, in the interest of conciseness and reducing the total number of figures, a given figure may be used to illustrate the features of more than one aspect of the disclosure, and not all elements in the figure may be required for a given aspect.

FIG. 1 is a block diagram of an example of utilizing an EM algorithm to determine a condition of sterilant to achieve an SAL for an article.

FIG. 2 is a block diagram of an example of utilizing a Bayesian MAP algorithm to determine a condition of sterilant to achieve an SAL for an article.

FIG. 3 is a diagram of an example of applying convergence criteria when utilizing the EM algorithm.

FIG. 4 is a diagram of an example of applying convergence criteria when utilizing the Bayesian MAP algorithm.

FIG. 5 is a block diagram of an example of a pilot study utilizing the EM algorithm to determine a condition of sterilant to achieve an SAL for an article.

FIG. 6 is a diagram of an example of an SAL curve that relates sterility assurance levels to units of sterilant.

FIG. 7 is a block diagram of an example of a pilot study utilizing a Bayesian MAP algorithm to determine a condition of sterilant to achieve an SAL for an article.

FIG. 8 is a diagram of a first example of applying a Bayesian MAP algorithm in a first iteration.

FIG. 9 is a diagram of a first example of applying a Bayesian MAP algorithm in a second iteration.

FIG. 10 is a diagram of a second example of applying a Bayesian MAP algorithm in a first iteration.

FIG. 11 is a diagram of a second example of applying a Bayesian MAP algorithm in a second iteration.

FIG. 12 is a block diagram of an example internal configuration of a computing device for determining a condition of sterilant to achieve an SAL for an article.

FIG. 13 is a flowchart of an example of a technique for determining a condition of sterilant to achieve an SAL for an article.

### DETAILED DESCRIPTION

There is a need to sterilize articles to an established minimum SAL. To do so, an article may be exposed to a sterilant under various conditions, such as an amount, an absorbed dose, and/or a duration of exposure, including at a given temperature, and/or pressure. It is therefore desirable to determine a least amount of sterilant and/or exposure for an article to achieve the minimum SAL. This may enable, for example, a reduction of waste associated with the sterilant, a faster sterilization of the article, enabling its sooner availability, and/or an improved life of the article by reducing wear associated with over-exposure. However, determining a minimum sterilant may be difficult, depending on several different factors including the SAL target for the article, the actual bioburden (e.g., a microbiology number) of the article, and the type of sterilant that is used.

In some cases, systems may attempt to determine an optimum amount of sterilant for an article by using a null hypothesis test (NHT). The NHT may consist of a classifier that estimates sterilant resistance to be greater than or less than a selected standard of resistance (Θ_{HS}). However, the NHT may also be inefficient to the extent that it does not consider how much greater than or less than the sterilant resistance may be relative to a selected standard of resistance (Θ_{HS}). This may result in extended testing to ensure a proper classification is achieved and/or an over-application of sterilant to achieve that classification. In other cases, systems may utilize different techniques that still result in an over-application of sterilant.

Implementations of this disclosure address problems such as these by utilizing machine learning algorithms to learn a microbiological quality and quantify a sterilant resistance of a microbial population. This may enable determining an application condition of sterilant to achieve an SAL for an article. In implementations, the one or more machine learning algorithms may include an EM algorithm and/or a Bayesian MAP algorithm. The algorithm, whether EM or Bayesian MAP, may be applied once or iteratively to estimate a value of a latent variable, e.g., Theta (Θ). In implementations, one of the algorithms can be used independently, e.g., the EM algorithm, or multiple algorithms can be combined, e.g., the EM algorithm and the Bayesian MAP algorithm used sequentially. The EM algorithm can generate an EM estimate which may be a maximum log likelihood estimate. The EM estimate may be analogous to a Bayesian MAP estimate. In implementations, the system can apply a single E-operation M-operation estimate, a multiple E-operation M-operation iteration estimate, a single operation Bayesian MAP estimate, a multiple operation Bayesian MAP estimate, or a combination thereof (e.g., a single operation Bayesian MAP estimate followed by a single E-operation M-operation estimate, a single E-operation M-operation estimate followed by a single operation Bayesian MAP estimate, or another sequence). As a result, the system can determine optimum application conditions of a sterilant to apply to an article to achieve an SAL. For example, the system can determine an amount, an absorbed dose, and/or a duration of exposure, including at a given temperature, and/or pressure, for a given sterilant modality to achieve a minimum SAL given a microbial resistance of an actual article.

In some implementations, an iterative convergence estimation may be utilized. For example, a system may receive input (e.g., user input through a graphical user interface) to define convergence criteria that identifies when a successful convergence is achieved. In some implementations, a number of trials, an initialization value, convergence criteria, and a number of iterations may be determined based on the input and its associated parameter estimation goal. In various implementations, a number of different sterilization modalities may be considered (e.g., radiation, Ethylene Oxide, dry heat, moist heat, or vaporized hydrogen peroxide). The algorithms described herein may be equally applicable to the different sterilization modalities and associated processes.

Several aspects of the disclosure with reference to the appended drawings are now explained. Whenever the shapes, relative positions and other aspects of the parts described are not explicitly defined, the scope of the invention is not limited only to the parts shown, which are meant merely for the purpose of illustration. Also, while numerous details are set forth, it is understood that some aspects of the disclosure may be practiced without these details. In other instances, well-known circuits, structures, and techniques have not been shown in detail so as not to obscure the understanding of this description.

Implementations disclosed herein provide a quantitative machine learning system that can generate a quantification of microbiological quality (Θ_{LV}) in the context of any hypothesized standard of sterilant resistance (Θ_{HS}). In various implementations, the system can eliminate or reduce imprecise indicator variables and/or confounding factors; quantify the probability of resistance of any microbial population (Θ_{LV}) in the context of any hypothesized (Θ_{HS}) standard of sterilant resistance, including for numerous sterilant modalities, such as radiation, Ethylene Oxide, dry heat, moist heat, and vaporized hydrogen peroxide; and/or estimate a quantity of sterilant and/or duration of exposure to a sterilant to achieve any user desired SAL in the context of the hypothesized sterilant resistance standard (Θ_{H}S).

FIG. 1 is a block diagram of an example of utilizing an EM algorithm 100 to determine a condition of sterilant to achieve an SAL for an article. The EM algorithm 100 may include the following operations: (1) initialization; (2) expectation (“E-operation”); and (3) maximization (“M-operation”). Iterations may comprise a repeated execution of the expectation and maximization operations. The acceptance criterion in each iteration may be that a fractional positive test of sterility (TOS) result is observed in the maximization operation(s). A stopping point may be identified when the result of the maximization operation achieves the convergence criteria. The fractional positive can also be expressed as a fractional negative.

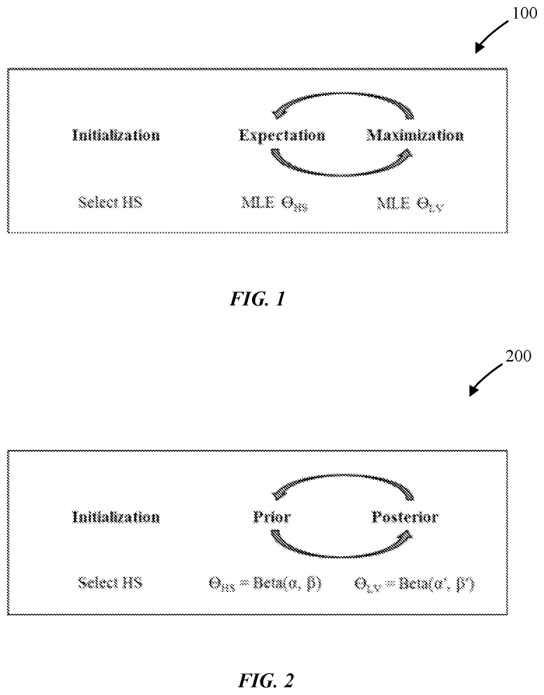

FIG. 2 is a block diagram of an example of utilizing a Bayesian MAP algorithm 200 to determine a condition of sterilant to achieve an SAL for an article. The Bayesian MAP algorithm 200 may include the following operations: (1) initialization; (2) prior; and (3) posterior. Thus, the Bayesian MAP algorithm 200 may comprise parallel operations of the EM algorithm 100, but with the E-operation and the M-operation corresponding to a prior operation and a posterior operation. Iterations may comprise a repeated execution of the prior and posterior operations.

In the EM algorithm 100 and the Bayesian MAP algorithm 200, the estimate of Θ_{LV} may be pulled into a subsequent iteration as either an expectation in the EM algorithm 100 or a prior of the Bayesian MAP algorithm 200. The EM algorithm 100 maximizes a likelihood (e.g., maximum likelihood estimate, MLE) and may be considered Frequentists methodology for point estimation of Θ_{L}V. The Bayesian MAP algorithm 200 maximizes a posterior mode and may be considered a Bayesian methodology that estimates a distribution of Θ_{LV}. The convergence criteria may define a stopping point, such as two iterations in examples described herein. In both algorithms, the size of the interval may be specified by user input as convergence criteria prior to executing the algorithm.

As described herein, a microbiological quality may be a descriptor of a population of micro-organisms where the descriptor is a quantified characterization of a population resistance (Θ_{LV}) in the context of a hypothesized resistance (Θ_{HS}). Historically, the descriptor of a population of micro-organisms in the context of a hypothesized resistance has been based on two indicators: a quantity of micro-organisms measured as colony forming unit (CFU), and a resistance (Θ_{HS}) of those micro-organisms to a sterilant. To emulate real-world populations, hypothesized resistances (Θ_{HS}) may comprise a weighted distribution of different resistances (e.g., Population C) as opposed to a singular resistance of a singular microbial species (e.g., a biological indicator). A CFU measurement is an indirect measurement of the size of a microbial population (e.g., a bioburden, or “bb”). The quantity of bioburden may be inferenced from a CFU indicator (e.g., bb=f (CFU)). This inference structure by design may result in an inferred quantity of micro-organisms based on a CFU enumerated count. In the case of a hypothesized weighted distribution of resistances the CFU enumeration may then be partitioned into sub-quantities as a proportion of the hypothesized resistance distribution weighting. Thus, the design premise is that any enumerated population (CFU) may be a precise estimate of the quantity of bioburden and is evaluated with an assumption of the hypothesized weighting of resistances (Θ_{HS}). However, an actual article's microbiological population might not be consistent in either the inferenced quantity (CFU) or assumed distribution weighting of resistance (Θ_{HS}), even between articles from a same manufacturing batch. This presents several problems in quantifying the microbiological quality.

It has been observed that the enumeration indicator value (CFU) to be a representation of the hypothesized weighted resistance distribution (Θ_{HS}) may be unfounded. This makes an enumerated value (CFU) an imprecise indicator of resistance. Further, it has been observed that the quality of the enumerated value (CFU) is confounded by several factors, e.g., test method recovery, default use of detection limit values, assumption of proportion weighting, dilution factors, and sample item portion (SIP) representation of device population. Additionally, it has been observed that the allowable variation between bioburden of individual samples inferenced from an enumerated count of cultured CFU can range up to 200% greater than the average in radiation sterilization dose establishment methods, or between 50% to 300% in the case of biological indicators used for other sterilant modalities, such as dry heat, moist heat, and Ethylene Oxide. Thus, implementations described herein including for radiation sterilization is eliminating the use of CFU enumeration (e.g., imprecise indicator/confounding factors).

Further, it has been observed that two prevalently used CFU-based methods for quantifying radiation resistance and the establishment of a sterilization dose, Method 1 and VDₘₐₓ, quantify sterilant resistance using an NHT. The NHT is limited in its ability to quantify, e.g., NHT represents a categorical classification. The NHT method simply indicates one of two classification categories, less than or equal to (<), or greater than (>), about a hypothesized theta (Θ_{HS}). Theta represents the probability of a sample expressing growth from a TOS evaluation (e.g., effectively the SAL). An additional limitation is that the NHT categorical classifier is premised on the inferred indicator value of bioburden (CFU). Thus, the result of either method (e.g., Method 1 or VDₘₐₓ) is that the forecast of the quantity of a sterilant to achieve a desired SAL is that of the hypothesized resistance standard (Θ_{HS}), not the actual article's microbiological population resistance (Θ_{LV}) under study. Another drawback of the NHT is that to the extent that the classification category is identified as greater than (>), the NHT result is not viable for forecasting (e.g., the NHT does not provide any forecast of resistance that can be leveraged to forecast a quantity of sterilant required for a targeted or desired SAL).

In various implementations, the system described herein provides one or more machine learning algorithms that learn (e.g., quantify) a resistance value theta (Θ_{LV}) of the article micro-organism population under study, whether a single species or resistance population or any weighted population permutation, as opposed to a categorical classification method. In various implementations, the algorithms may avoid an inferred enumeration value (CFU), thereby eliminating less desirable indicator variables and their confounders. For example, the algorithms described herein may generate results that are an estimation of a statistical distribution of the parameter Θ_{LV} in the case of the Bayesian MAP algorithm 200, or a point estimate of the value of Θ_{LV} in the case of the EM algorithm 100, as opposed to categorizing a result. The maximization estimate of Θ_{LV} (MLE or MAP) comprises a machine learning structure that is a modification of belief as a result of evidence.

### Quantifying Probability of Microbial Resistance Using EM (Θ_{LV})

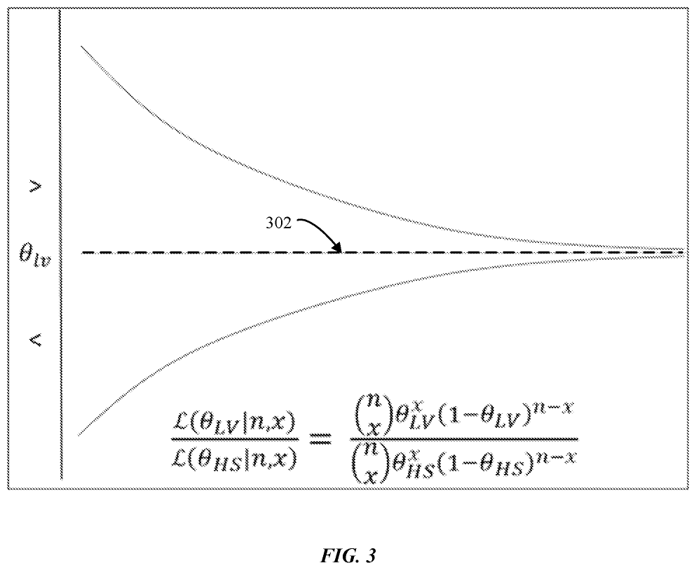

With additional reference to FIG. 3, the EM algorithm 100 may generate an estimation of a latent variables distribution parameter Θ_{LV} based on the initialization operation selection of a resistance standard (HS) and the expectation operation MILE Θ_{HS}. The examples described herein include two iterations of the EM algorithm 100. The EM algorithm 100 iteration is an iteration of the E-operation and M-operation pair until convergence is achieved. For example, the system may receive input (e.g., user input through a graphical user interface) to define convergence criteria that identifies when a successful convergence is achieved, such as a point 302 between upper and lower limits of Θ_{LV}.

The initialization value of the latent variable may be taken as a user selection of a resistance standard (e.g., user input). In the examples presented herein, the latent variable may be that of a SAL estimate of a standard distribution of resistance (SDR) (e.g., Population C) sterility assurance level for a single article for a selected dose (e.g., the sterilant in this example being radiation in kGy).

The E-operation may comprise estimating the expression of the latent variable (fractional positives) based on the initialization selection of a resistance standard, quantity and/or duration of exposure to a sterilant, the corresponding Θ_{HS} of the resistance standard, and the number of samples that provide the basis of expectation of fractional positives of the data. The likelihood for the resistance standard selected and quantity of sterilant or sterilant process exposure may be forecast directly from the HS resistance standard SAL-relationship. Given a specified number of samples, the expectation of fractional positives may be defined.

The M-operation may comprise exposing the defined number of samples to the defined quantity of sterilant or a duration of sterilant exposure and submitting the samples to a TOS. For example, the samples may be obtained from a same manufacturing batch. In some cases, the samples may be obtained from different manufacturing batches. The TOS results may be expressed as fractional positives for growth that follow a binomial distribution. A maximum likelihood estimate (MILE) is prepared from the TOS for Θ_{L}V. An assessment of the result may be calculated as a ratio of maximum likelihoods, reference Eq. 1.

$$\frac{ℒ ( {θ}_{lv} \vert n , x )}{ℒ ( {θ}_{HS} \vert n , x )} = \frac{( \begin{matrix}n \\ x\end{matrix} ) {{θ}_{lv}^{x} ( 1 - {θ}_{lv} )}^{n - x}}{( \begin{matrix}n \\ x\end{matrix} ) {{θ}_{HS}^{x} ( 1 - {θ}_{HS} )}^{n - x}} \qquad Eq . 1$$

The likelihood for the result of the TOS (Θ_{LV}) may have the generalized result of x/n. This ratio can be a large number or a small number. Once the MILE Θ_{L}V is estimated, convergence analysis may be performed to determine whether a user defined convergence threshold has been achieved (e.g., based on the input).

Performance of a successful M-operation may be based on a fractional positive being observed. If a fractional positive is not observed, the MLE Θ_{L}V is either zero or 1.0. In the case of zero, the E-operation selection of the quantity of sterilant or sterilant process exposure may be too great. This may indicate the actual microbiological resistance population of the article is lower than Θ_{HS}, and the quantity of sterilant or sterilant process exposure may be reduced in a subsequent iteration. In some cases, this can also represent a special case called zero-inflation which is observed when establishing that an article is sterile as manufactured using Laplace smoothing. In the case of 1.0, the E-operation selection of the quantity of sterilant or sterilant process exposure may be too small. This may indicate the actual microbiological resistance population of the article is greater than Θ_{HS}, and the quantity of sterilant or sterilant process exposure may be increased in a subsequent iteration.

When the ratio estimate in Equation 1 is significantly larger than 1 or significantly less than 1, the E-operation estimate of Θ_{HS} may be rejected. The ratio value of Equation 1 may be an estimate of bias between the selected resistance standard (Θ_{HS}) and the actual article resistance (Θ_{LV}). The estimate of bias may be applied as a correction to the resistance standard SAL-curve (e.g., the basis of deriving Θ_{HS}). The corrected SAL-curve then is the basis for the next E-operation iteration. The bias correction may represent a vertical shift up or a vertical shift down of the resistance standard SAL-curve and Θ_{HS} y-intercept determined in the E-operation.

### Quantifying Probability of Microbial Resistance Using Bayesian MAP (Θ_{LV})

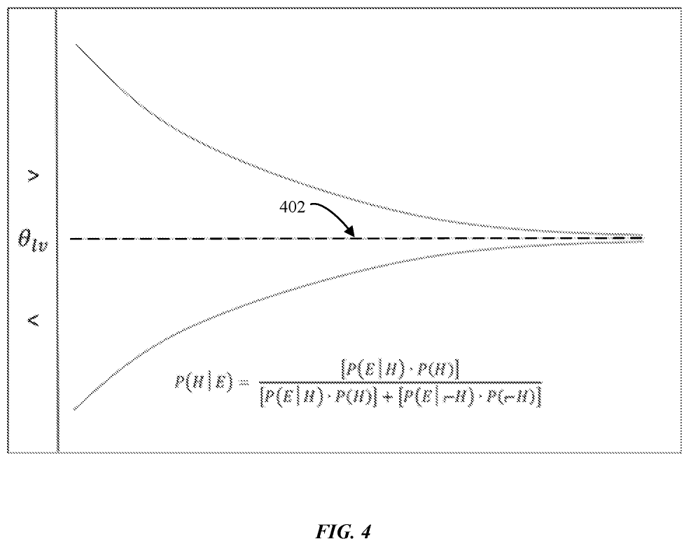

With additional reference to FIG. 4, the Bayesian MAP algorithm 200 may generate an estimation of a latent variables distribution parameter Θ_{LV} based on the initialization operation selection of a resistance standard (HS) and the prior operation Θ_{HS}=Beta (α, β). The proposed MLLP Θ_{L}V innovation examples in this document are shown with two iterations of the MAP algorithm. The MAP algorithm iteration is an iteration of the prior-operation and posterior-operation pair until convergence is achieved, reference FIG. 2. For example, the system may receive input (e.g., user input through a graphical user interface) to define convergence criteria that identifies when a successful convergence is achieved, such as a point 402 between upper and lower limits of Θ_{LV}.

The initialization value of the latent variable may be taken as a user selection of a resistance standard (e.g., user input). In the examples presented herein, the latent variable may be that of a SAL estimate of an SDR (e.g., Population C) sterility assurance level for a single article for a selected dose (e.g., the sterilant in this example being radiation in kGy).

The Prior-operation may comprise estimating the expression of the latent variable (fractional positives) based on the initialization selection of a resistance standard, quantity and/or duration of exposure to a sterilant, the corresponding Θ_{HS} of the resistance standard, and the number of samples that provide the basis of expectation of fractional positives of the data. The likelihood for the resistance standard selected and quantity of sterilant or sterilant process exposure may be forecast directly from the HS resistance standard SAL-relationship. Given a specified number of samples, the expectation of fractional positives may be defined.

The Posterior-operation may comprise exposing the defined number of samples to the defined quantity of sterilant or a duration of sterilant exposure and submitting the samples to a TOS. The TOS results may be expressed as fractional positives for growth that follow a binomial distribution. A maximum a posteriori estimate (MAP) is prepared from the TOS for Θ_{LV}. An assessment of the result may be calculated as a ratio of maximum a posteriori, reference Eq. 2.

$$P ( H \vert E ) = \frac{[ P ( E \vert H ) \cdot P ( H ) ]}{[ P ( E \vert H ) \cdot P ( H ) ] + [ P ( E \vert H ) \cdot P ( H ) ]} \qquad Eq . 2$$

The MAP for the result of the TOS may have the generalized distribution form of Beta (α′, β′). The mode of the Beta distribution is the MAP Θ_{LV}. A ratio of Θ_{LV} to Θ_{HS} is calculated. The ratio can be a large number or a small number. Once the MAP Θ_{LV} is estimated, convergence analysis may be performed to determine whether a user defined convergence threshold has been achieved (e.g., based on the input).

Performance of a successful posterior-operation may be based on a fractional positive being observed. If a fractional positive is not observed, the MAP Θ_{LV} is either zero or 1.0. In the case of zero, the prior-operation selection of the quantity of sterilant or sterilant process exposure may be too great. This may indicate the actual microbiological resistance population of the article is lower than Θ_{HS}, and the quantity of sterilant or sterilant process exposure may be reduced in a subsequent iteration. In some cases, this can also represent a special case called zero-inflation which is observed when establishing that an article is sterile as manufactured using Zero-Inflated Beta Prior. In the case of 1.0, the prior-operation selection of the quantity of sterilant or sterilant process exposure may be too small. This may indicate the actual microbiological resistance population of the article is greater than Θ_{HS}, and the quantity of sterilant or sterilant process exposure may be increased in a subsequent iteration.

When the ratio estimate of MAP Θ_{LV} to MAP Θ_{HS} is significantly larger than 1 or significantly less than 1, the prior-operation estimate of Θ_{HS} may be rejected. The ratio value may be an estimate of bias between the selected resistance standard (Θ_{HS}) and the actual article resistance (Θ_{LV}). The estimate of bias may be applied as a correction to the resistance standard SAL-curve (e.g., the basis of deriving Θ_{HS} and the Beta prior associated with the new bias corrected Θ_{HS}). The bias correction may represent a vertical shift up or a vertical shift down of the resistance standard SAL-curve and Θ_{HS} y-intercept determined in the prior-operation.

### Pilot Study 1: Radiation Computational Example Using EM

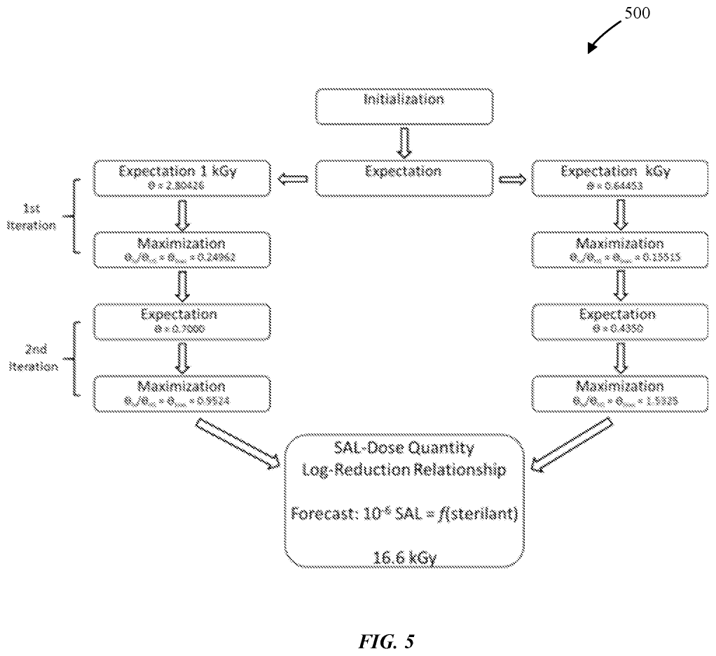

FIG. 5 is a block diagram of an example of a pilot study 500, utilizing the EM algorithm 100, to determine a condition of sterilant (e.g., 16.6 KGy, with the sterilant in this example being radiation) to achieve an SAL (e.g., 10⁻⁶) for an article. The pilot study 500 was performed in parallel with a VDₘₐₓ dose establishment method so that the results of implementations described herein could be compared directly to an existing bioburden-based method.

### Initialization

The initialization operation of the pilot study 500 identifies the resistance standard to be used. The standard distribution of resistance (SDR) Population C of the ISO 11137-2 Method 1 was selected. This resistance standard was selected based on (1) long standing use and explicitly defined weighting of resistance; and extensive tables describing the resistance for a wide range of doses and quantities of bioburden (CFU).

### E-Operation/First Iteration.

The E-operation defines an expected expression of the test variable (fractional positives) and an inferenced estimate of the latent variable parameter Θ_{HS} of the resistance standard selected. Three sample groups of 10 samples each of an article were prepared and each group was targeted with one of three dose magnitudes. Three dose magnitudes were selected to ensure at least two groups would express fractional positive in a TOS. However, this is not a requirement for the proposed method. The use of three groups was to ensure at least two groups with fractional positives would be expressed so that convergence of two different values of fractional positives could be evaluated. In various implementations, a single sample group that expresses a fractional positive may be used.

Target doses of 1 kGy, 2 kGy, and 3 kGy were selected for each of the three groups ofZ 10 samples, respectively. These selected target doses afforded a high likelihood that at least two fractional positives would be observed and with adequate spacing so that a ±20% dose range for any sample group would not over-lap an adjacent target dose group.

### M-Operation/First Iteration.

Samples were irradiated in a single plane to ensure a smallest range of dose was delivered to each sample group. Dosimetry was placed on the sample array to measure dose delivery. Following irradiation, samples were placed on a growth media and incubated for 14 days. At the end of incubation, samples expressing growth were counted and fractional positives for each group quantified. See Table 1 for fractional positive results.

TABLE 1
Fractional Positive Expression Results

| Sample Group | 1 kGy | 2 kGy | 3 kGy |
|---|---|---|---|
| Proportion of Samples | 7/10 | 1/10 | 0/0 |
| Expressing Growth | | | |

### 1 kGy Fractional Positive Group

A fractional positive of 7 out of 10 samples was observed. This result indicates a maximum likelihood value of the latent variable Θ_{LV} of 0.7. The ratio of the observed fractional positive Θ_{LV} to the SDR forecast Θ_{HS} results in a bias estimate of 0.24962 under the assumption of a 21 CFU Θ_{HS}. This correction was applied to the Θ_{HS} SAL to dose curve dependent variable and regressed over the domain values of 0 kGy, 5 kGy, 11 kGy, 14.2 kGy, 17.6 kGy, 21.2 kGy, 24.9 kGy and 30 kGy using Equation 3. The regressed values are shown in Table 2.

$$ln y = ( a + cx + {ex}^{2} ) / ( 1 + bx + {dx}^{2} ) \qquad Eq . 3$$

TABLE 2
Adjusted resistance curve

| Dose, | | | | | | | | |
|---|---|---|---|---|---|---|---|---|
| kGy | 0 | 5 | 11 | 14.2 | 17.6 | 21.2 | 24.9 | 30 |
| SAL | 5.242 | 6.4581e−3 | 5.1374e−5 | 5.2825e−6 | 5.3336e−7 | 5.1943e−8 | 5.1132e−9 | 2.2823e−10 |

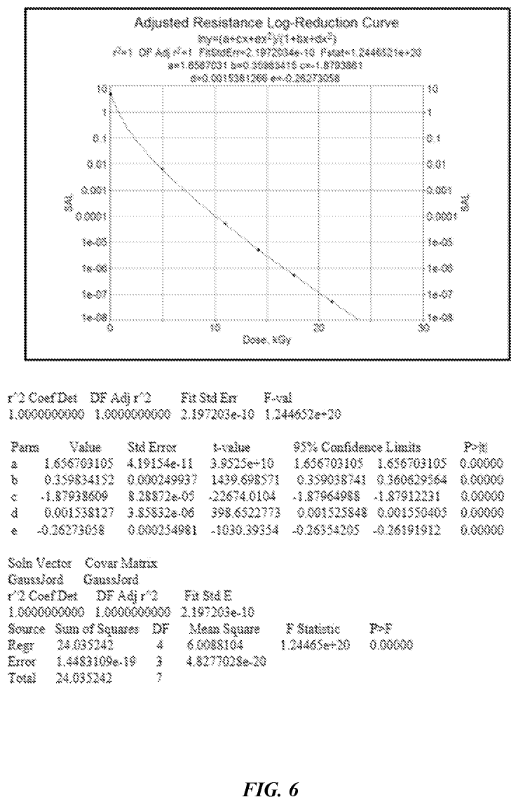

With additional reference to FIG. 6, a bias corrected SAL to dose curve (e.g., which relates sterility assurance levels to units of sterilant) is then carried forward into the E-operation of the next iteration for forecasting a dose, fractional positive, and corresponding latent SAL Θ_{HS}. This completes the first iteration of the EM algorithm 100.

It is noteworthy that the assumed CFU of the Θ_{HS} in this first M-operation result is inconsequential to the final result of the SAL curve correction given the observed likelihood (0.7) from the 1 kGy exposure. As an example, Table 3 shows various assumed CFU values, the bias estimate of the likelihood ration, and the bias correction applied to estimate the y-intercept of the bias adjusted SAL curve (e.g., the vertical shift of the Θ_{HS} SAL to dose curve). Any assumed CFU forecasts the same new bias adjusted SAL to dose curve y-intercept to within a few thousandths given the observed likelihood. Thus, the only stipulated acceptance requirement of the M-operation is that a fractional positive result is observed.

TABLE 3
Convergence of Likelihood Ratio
Correction Estimate on y-intercept

| | | Assumed | Likeli- | |
|---|---|---|---|---|
| | Observed | Latent | hood | LR * |
| Assumed | Likeli- | Likeli- | Ratio | Assumed CFU |
| CFU | hood | hood | (LR) | (y-intercept) |
| 1 CFU | 0.7 | 0.13355 | 5.2414 | 5.241 |
| 2 CFU | 0.7 | 0.26717 | 2.62005 | 5.240 |
| 5 CFU | 0.7 | 0.66772 | 1.04834 | 5.242 |
| 10 CFU | 0.7 | 1.3352 | 0.52426 | 5.243 |
| 20 CFU | 0.7 | 2.6717 | 0.26200 | 5.240 |
| 50 CFU | 0.7 | 6.6772 | 0.10483 | 5.242 |
| 100 CFU | 0.7 | 13.352 | 0.05242 | 5.242 |
| 800 CFU | 0.7 | 106.83 | 0.00655 | 5.240 |
| 1500 CFU | 0.7 | 200.19 | 0.00349 | 5.235 |

### 1 kGy E-Operation/Second Iteration

The second iteration E-operation was designed to (1) provide improved resolution (e.g., hyper-tune) of the Θ_{LV} value estimate by using 30 samples as opposed to the 10 samples of the first iteration (e.g., 0.03 increments, as opposed to 0.10 increments); and (2) provide an estimate of the Θ_{LV} value over several (e.g., 3) manufacturing batches, thereby ensuring any microbiological quality variability in manufacturing batch to manufacturing batch was accounted for in the Θ_{LV} estimate.

Ten samples each of an article from three manufacturing batches were selected to provide a total of 30 samples. A target dose of approximately 1.2 kGy was selected to provide an estimated value of the latent variable parameter Θ_{HS}=0.5059. The targeted latent variable parameter of Θ_{HS}=0.5059 enables hyper-tuning above 0.5059 and below 0.5059. Note, any value within the range of 0.20 to 0.80 (0.5059 was selected) may be suitable as this range would account for any sampling error resolution in the first M-operation iteration for potential significant variation in microbiological quality manufacturing batch to manufacturing batch, e.g., a variation with a factor of two greater than was observed within a single manufacturing batch. On the outside chance that a factor greater than 2 were to express, the targeting of Θ_{HS}=0.5059 can accommodate a larger variance than was observed in the first EM iteration.

### 1 kGy M-Operation/Second Iteration

Samples were irradiated targeting a dose of 1.2 kGy in a single plane to ensure a smallest range of dose was delivered to the sample group. Dosimetry was placed on the sample array to measure dose delivery. The achieved dose was 1.0 kGy. As a result, an updated Θ_{HS} value was calculated based on the previous M-operation SAL to dose curve result for the achieved 1.0 kGy dose. The updated Θ_{HS} was 0.7000. Following irradiation, samples were placed on a growth media and incubated for 14 days (TOS). At the end of incubation, samples expressing growth were counted and fractional positives for each group quantified. A fractional positive result of 20 out of 30 samples was observed. See Table 4 for fractional positive results.

TABLE 4
M-operation fractional positives for second M-operation iteration

| | | | Observed |
|---|---|---|---|
| Healthcare | Targeted Dose (kGy) | Achieved | Fractional |
| Product | based on | Dose | Positive |
| Batch | corrected Θ_{HS} | (kGy) | of TOS |
| AT6615 | 1.2 | 1.0 | 7/10 |
| AT8450 | 1.2 | 1.0 | 6/10 |
| AT8451 | 1.2 | 1.0 | 7/10 |

This result indicates a maximum likelihood value of the latent variable Θ_{L}V of 0.6667. The ratio of the observed fractional positive Θ_{L}V to the forecast Θ_{HS} of 0.7000 resulted in a bias estimate of 0.9524 under the ‘assumption’ of a 5.242 CFU Θ_{HS}. The correction resulted in a y-intercept value of 4.992. The correction was applied to the Θ_{HS} SAL to dose curve and a final bias corrected SAL to dose curve was calculated, reference Table 5.

TABLE 5
Article microbial resistance curve θ_{LV}

| | Dose, kGy | | | | | | | |
|---|---|---|---|---|---|---|---|---|
| | 0 | 5 | 11 | 14.2 | 17.6 | 21.2 | 24.9 | 30 |
| SAL | 4.992 | 6.1506e⁻³ | 4.8928e⁻⁵ | 5.0310e⁻⁶ | 5.0797e⁻⁷ | 4.9470e⁻⁸ | 4.8698e⁻⁹ | 2.1736e⁻¹⁰ |

This final corrected SAL to dose curve estimates the SAL doses shown in Table 6.

TABLE 6
Dose to SAL

| SAL | 10⁻¹ | 10⁻² | 10⁻³ | 10⁻⁴ | 10⁻⁵ | 10⁻⁶ |
|---|---|---|---|---|---|---|
| Dose, | 2.32 | 4.48 | 7.10 | 10.04 | 13.21 | 16.58 |
| kGy | | | | | | |

### 2 kGy Fractional Positive Group

A fractional positive of 1 out of 10 samples was observed. This result indicates a maximum likelihood value of the latent variable Θ_{L}V of 0.1. The ratio of the observed fractional positive Θ_{L}V to the SDR forecast Θ_{HS} results in a bias estimate of 0.15515 under the assumption of a 21 CFU Θ_{HS}. This correction was applied to the Θ_{HS} and a bias corrected SAL to dose curve was calculated, reference Table 7.

TABLE 7
Adjusted resistance curve

| | Dose, kGy | | | | | | | |
|---|---|---|---|---|---|---|---|---|
| | 0 | 5 | 11 | 14.2 | 17.6 | 21.2 | 24.9 | 30 |
| SAL | 3.258 | 4.0142e⁻³ | 3.1933e⁻⁵ | 3.2834e⁻⁶ | 3.3152e⁻⁷ | 3.2287e⁻⁸ | 3.1782e⁻⁹ | 1.4186e⁻¹⁰ |

The bias corrected SAL to dose curve is then carried forward into the E-operation of the next iteration for forecasting a dose, fractional positive, and the corresponding latent SAL Θ_{HS}. It is noteworthy that the assumed CFU of the Θ_{HS} in this first M-operation result is inconsequential to the final result of the SAL curve correction given the observed likelihood (0.1) from the 2 kGy exposure. As an example, Table 8 shows various assumed CFU values, the bias estimate of the likelihood ratio, and the correct first dose level of the adjusted SAL curve (e.g., the vertical shift of the Θ_{HS} SAL to dose curve). Any assumed CFU forecasts the same new adjusted SAL to dose curve y-intercept to a few thousandths. This supports the only stipulated acceptance requirement of the M-operation is that a fractional positive result is observed.

TABLE 8
Convergence of Likelihood Ratio Correction Estimate on CFU

| | | Assumed | | LR * Assumed |
|---|---|---|---|---|
| Assumed | Observed | Latent | Likelihood | CFU |
| CFU | Likelihood | Likelihood | Ratio (LR) | (y-intercept) |
| 1 CFU | 0.1 | 0.03069 | 3.2583 | 3.258 |
| 2 CFU | 0.1 | 0.06140 | 1.62866 | 3.257 |
| 5 CFU | 0.1 | 0.15346 | 0.65163 | 3.258 |
| 10 CFU | 0.1 | 0.30695 | 0.32578 | 3.258 |
| 20 CFU | 0.1 | 0.6140 | 0.16286 | 3.257 |
| 50 CFU | 0.1 | 1.5346 | 0.06516 | 3.258 |
| 100 CFU | 0.1 | 3.0695 | 0.03257 | 3.257 |
| 800 CFU | 0.1 | 24.553 | 0.00407 | 3.257 |
| 1500 CFU | 0.1 | 46.025 | 0.00217 | 3.255 |

This completes the first iteration of the EM algorithm 100.

### 2 kGy: E-Operation/Second Iteration

The second iteration E-operation was designed to (1) provide improved resolution (e.g., hyper-tune) of the Θ_{L}V value estimate by using 30 samples as opposed to the 10 samples of the first iteration (e.g., 0.03 increments, as opposed to 0.10 increments); and (2) provide an estimate of the Θ_{L}V value over several (e.g., 3) manufacturing batches, thereby ensuring any microbiological quality variability in manufacturing batch to manufacturing batch was accounted for in the Θ_{LV} estimate.

Ten samples each of an article from three manufacturing batches were selected to provide a total of 30 samples. A target dose of approximately 0.92 kGy was selected to provide an estimated value of the latent variable parameter Θ_{HS}=0.50. The targeted latent variable parameter of Θ_{HS}=0.50 enables hyper-tuning above 0.50 and below 0.50. Note, any value within the range of 0.20 to 0.80 may be suitable as this range would account for any sampling error resolution in the first M-operation iteration for potential significant variation in microbiological quality manufacturing batch to manufacturing batch, e.g., a variation with a factor of two greater than was observed within a single manufacturing batch. On the outside chance that a factor greater than two were to express, the targeting of Θ_{HS}=0.50 can accommodate a larger variance than was observed in the first EM iteration.

### 2 kGy M-Operation/Second Iteration

Samples were irradiated targeting a dose of 0.92 kGy in a single plane to ensure a smallest range of dose was delivered to the sample group. Dosimetry was placed on the sample array to measure dose delivery. The achieved dose was 1.0 kGy. As a result, an updated Θ_{HS} value was calculated based on the previous M-operation SAL to dose curve result for the achieved 1.0 kGy dose. The updated Θ_{HS} was 0.4350. Following irradiation, samples were placed on a growth media and incubated for 14 days. At the end of incubation, samples expressing growth were counted and fractional positives for each group quantified. A fractional positive result of 20 out of 30 samples was observed. See Table 9 for fractional positive results.

TABLE 9
M-operation fractional positives for second M-operation iteration

| | Targeted Dose | | Observed |
|---|---|---|---|
| Healthcare | (kGy) based | Achieved | Fractional |
| Product | on corrected | Dose | Positive |
| Batch | Θ_{HS} | (kGy) | of TOS |
| AT6615 | 1.2 | 1.0 | 7/10 |
| AT8450 | 1.2 | 1.0 | 6/10 |
| AT8451 | 1.2 | 1.0 | 7/10 |

This result indicates a maximum likelihood value of the latent variable Θ_{LV} of 0.6667. The ratio of the observed fractional positive Θ_{LV} to the forecast Θ_{HS} of 0.4350 results in a bias estimate of 1.5325 under the assumption of a 3.258 CFU Θ_{HS}. The correction results in a y-intercept value of 4.992. The correction was applied to the Θ_{HS} SAL to dose curve and a final bias corrected SAL to dose curve was calculated, reference Table 10.

TABLE 10
healthcare product microbial resistance curve θ_{LV}

| | Dose, kGy | | | | | | | |
|---|---|---|---|---|---|---|---|---|
| | 0 | 5 | 11 | 14.2 | 17.6 | 21.2 | 24.9 | 30 |
| SAL | 4.992 | 6.1517e−3 | 4.8937e−5 | 5.0318e−6 | 5.0805e−7 | 4.9479e−8 | 4.8705e−9 | 2.1740e−10 |

This final corrected SAL to dose curve estimates the SAL doses shown in Table 11.

TABLE 11
Dose to SAL

| SAL | 10⁻¹ | 10⁻² | 10⁻³ | 10⁻⁴ | 10⁻⁵ | 10⁻⁶ |
|---|---|---|---|---|---|---|
| Dose, | 2.34 | 4.48 | 7.09 | 10.03 | 13.21 | 16.58 |
| kGy | | | | | | |

A comparison of the EM algorithm 100 result for both the initial 0.7 fractional positive and 0.1 fractional estimates of healthcare product microbial resistance is shown in Table 12. Both derivations of the healthcare product microbial resistance provide nearly identical estimates of various SAL doses within two decimal places, e.g., to 1/100 of a kGy.

TABLE 12
Hyper-tune residual comparison of product
microbial resistance curves

| | Initial 1 | Initial 2 | |
|---|---|---|---|
| SAL | kGy Pilot | kGy Pilot | Difference |
| 10⁻¹ | 2.33 kGy | 2.34 kGy | 0.01 kGy |
| 10⁻² | 4.48 kGy | 4.48 kGy | 0.00 kGy |
| 10⁻³ | 7.09 kGy | 7.09 kGy | 0.00 kGy |
| 10⁻⁴ | 10.03 kGy | 10.03 kGy | 0.00 kGy |
| 10⁻⁵ | 13.21 kGy | 13.21 kGy | 0.00 kGy |
| 10⁻⁶ | 16.58 kGy | 16.58 kGy | 0.00 kGy |

Table 12 demonstrates that the magnitude of the fractional positive identified in the first iteration of the E-operation and M-operation has no impact on the EM algorithm 100. Any fractional positive result will ultimately arrive as the same SAL to sterilant relationship, e.g., the actual article microbiological quality and log-reduction curve.

### Parallel Study Using VDₘₐₓ 22.5 Dose Establishment Method

In parallel to the pilot study 500, a VDₘₐₓ 22.5 dose establishment method was performed. The summary results of the VDₘₐₓ 22.5 method are shown in the Table 13 below. The results indicate a successful validation of the VDₘₐₓ 22.5 kGy dose.

TABLE 13
Results of a VDₘₐₓ 22.5 Validation—
Healthcare Product VR 932

| | Bioburden | | Target | Achieved | Test of |
|---|---|---|---|---|---|
| | (CFU/ | Average | Radiation | Radiation | Sterility |
| Batch | product) | Bioburden | Dose | Dose | Result |
| AT6615 | 1 | 8 CFU | 6.2 kGy | 6.1 kGy | 0/10 |
| AT8450 | 11 | | | | |
| AT8451 | 11 | | | | |

### Pilot Study 2: Radiation Computational Example Using Bayesian MAP

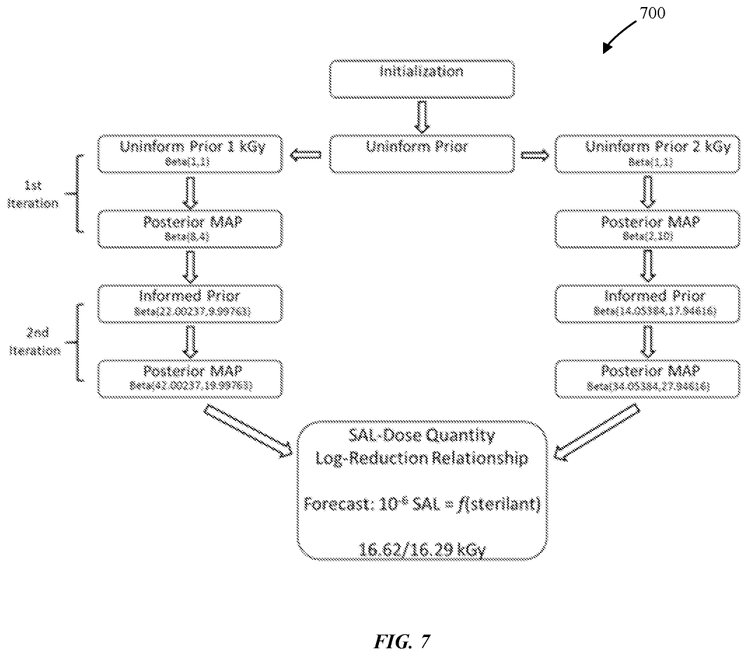

FIG. 7 is a block diagram of an example of a pilot study 700, utilizing the Bayesian MAP algorithm 200, to determine a condition of sterilant (e.g., 16.62/16.29 KGy, with the sterilant in this example being radiation) to achieve an SAL (e.g., 10⁻⁶) for an article. The pilot study 700 represents a parallel computation of the pilot study 500 (e.g., 1 kGy and 2 kGy pilot results), now using the Bayesian MAP algorithm 200 to estimate of Θ_{L}V and correction of Θ_{HS}.

### 1 kGy Prior-Operation/Posterior-Operation/First Iteration

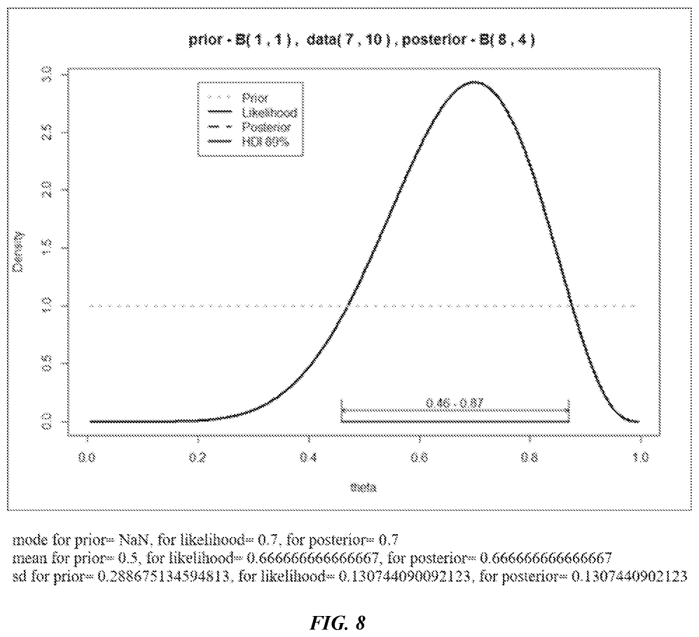

FIG. 8 is a diagram of a first example (e.g., 1 kGy) of applying the Bayesian MAP algorithm 200 in a first iteration using an uninformed prior.

### 1 kGy Prior-Operation/Posterior-Operation/Second Iteration

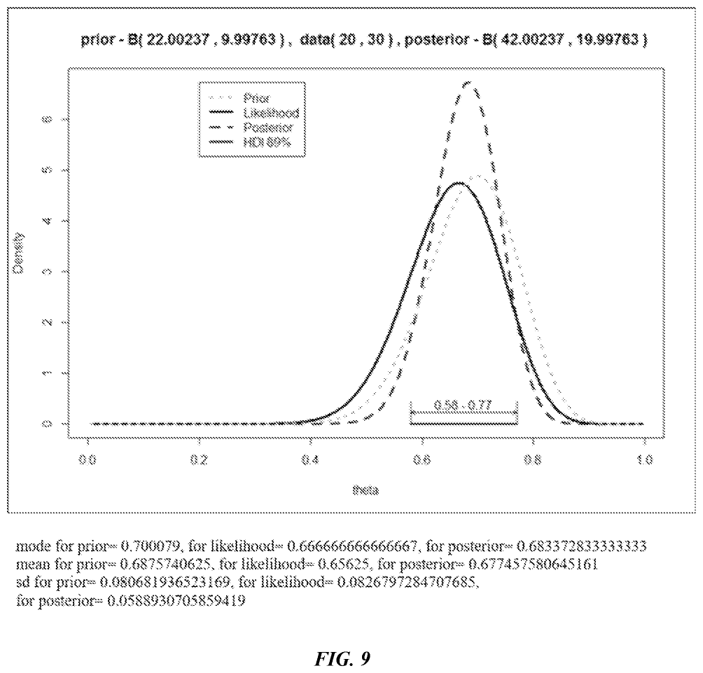

FIG. 9 is a diagram of the first example (e.g., 1 kGy) of applying the Bayesian MAP algorithm 200 in a second iteration using an informed prior. The MAP method for derivation of Theta used to correct assumed resistance model is 0.6833728. This is then used in the ratio to correct assumed resistance model. This value gives a correction value of 0.976136 or a y-intercept of 5.116. The adjusted SAL to Dose curve is then given by:

TABLE 14
Healthcare product microbial resistance curve θ_{LV}

| | Dose, kGy | | | | | | | |
|---|---|---|---|---|---|---|---|---|
| | 0 | 5 | 11 | 14.2 | 17.6 | 21.2 | 24.9 | 30 |
| SAL | 5.116 | 6.30398e−3 | 5.0148e−5 | 5.1564e−6 | 5.2063e−7 | 5.0703e−8 | 4.9912e−9 | 2.2278e−10 |

This SAL to Dose curve may forecast:

TABLE 15
Dose to SAL

| SAL | 10⁻¹ | 10⁻² | 10⁻³ | 10⁻⁴ | 10⁻⁵ | 10⁻⁶ |
|---|---|---|---|---|---|---|
| Dose, | 2.34 | 4.51 | 7.13 | 10.07 | 13.25 | 16.62 |
| kGy | | | | | | |

### 2 kGy Prior-Operation/Posterior-Operation/First Iteration

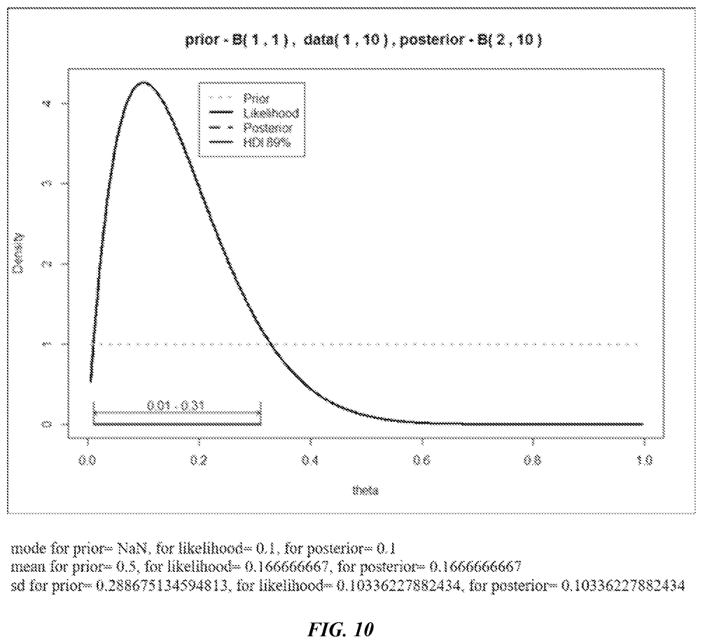

FIG. 10 is a diagram of a second example (e.g., 2 kGy) of applying the Bayesian MAP algorithm 200 in a first iteration using an uninformed prior.

### 2 kGy Prior-Operation/Posterior-Operation/Second Iteration

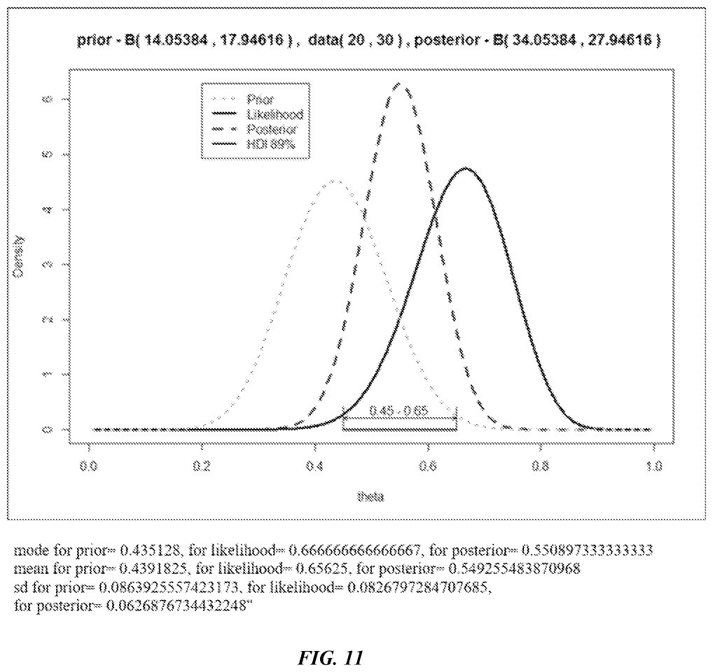

FIG. 11 is a diagram of the second example (e.g., 2 kGy) of applying the Bayesian MAP algorithm 200 in a second iteration using an informed prior. The adjusted SAL to Dose curve is then given by:

TABLE 16
healthcare product microbial resistance curve θ_{LV}

| | Dose, kGy | | | | | | | |
|---|---|---|---|---|---|---|---|---|
| | 0 | 5 | 11 | 14.2 | 17.6 | 21.2 | 24.9 | 30 |
| SAL | 4.125 | 5.08197e−3 | 4.0427e−5 | 4.1569e−6 | 4.1971e−7 | 4.0875e−8 | 4.0237e−9 | 1.7960e−10 |

This SAL to Dose curve may forecast:

TABLE 17
Dose to SAL

| SAL | 10⁻¹ | 10⁻² | 10⁻³ | 10⁻⁴ | 10⁻⁵ | 10⁻⁶ |
|---|---|---|---|---|---|---|
| Dose, | 2.18 | 4.28 | 6.86 | 9.78 | 12.94 | 16.29 |
| kGy | | | | | | |

Thus, a system that implements the one or more machine learning algorithms described herein (e.g., the EM algorithm 100 and/or the Bayesian MAP algorithm 200) may provide several improvements. For example, the system may reduce or eliminate dependence on a CFU inference of bioburden quantity, thus eliminating confounding factors and inefficiencies associated with this indicator. The system may also provide a 10⁻⁶ SAL estimate or any other desired SAL of the actual article sterilant resistance (microbiological quality), as opposed to a default resistance of a standard. The system can be applied to any sterilant resistance standard. The system in almost all cases may provide an estimate of a 10⁻⁶ SAL quantity of sterilant that is less than predominantly used methods (e.g., Method 1 and VDₘₐₓ), resulting in unrealized sterilization capacity and reducing sterilant degradation of healthcare products functionality or potency. As it is based on a machine learning (e.g., EM or Bayesian MAP), several pathways to validating an article (e.g., a healthcare product) is sterile as manufactured can be employed for, e.g., the EM algorithm 100 using Laplace smoothing for zero-inflation, or a Zero-Inflated Beta Prior in the case of the Bayesian MAP algorithm 200. As a measurement of microbiological quality, this can be used in the statistical modeling and assessment of healthcare product manufacturing processes, e.g., statistical modeling of at each manufacturing step to identify manufacturing steps that represent dominant contributing factors of a healthcare product microbiological quality. The system can also be used to standardize manufactured microbial resistance standards (e.g., Biological Indicator employed in gaseous or heat sterilization).

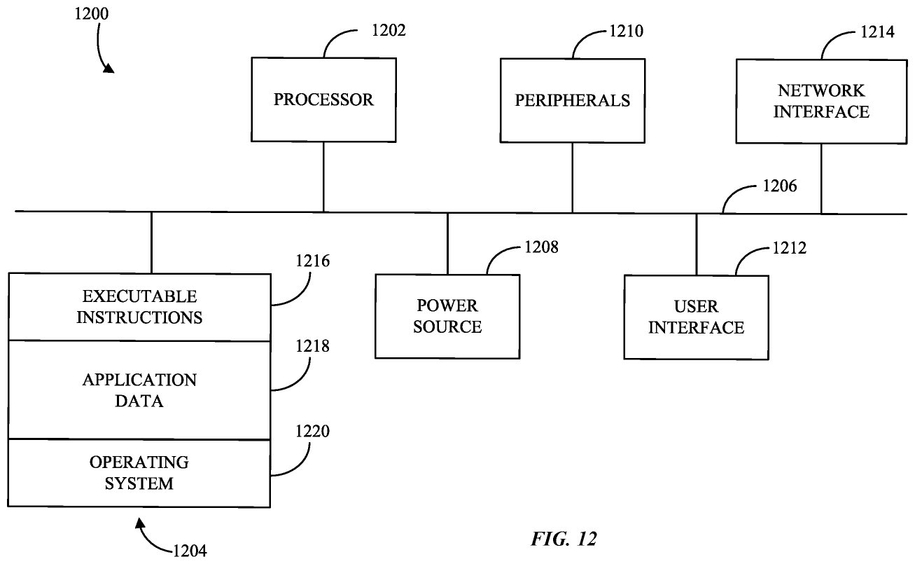

FIG. 12 is a block diagram of an example internal configuration of a computing device 1200 for determining a condition of sterilant to achieve an SAL for an article. In one configuration, the computing device 1200 may comprise an apparatus configured to implement the EM algorithm 100 and/or the Bayesian MAP algorithm 200.

The computing device 1200 includes components or units, such as a processor 1202, a memory 1204, a bus 1206, a power source 1208, peripherals 1210, a user interface 1212, a network interface 1214, other suitable components, or a combination thereof. One or more of the memory 1204, the power source 1208, the peripherals 1210, the user interface 1212, or the network interface 1214 can communicate with the processor 1202 via the bus 1206.

The processor 1202 is a central processing unit, such as a microprocessor, and can include single or multiple processors having single or multiple processing cores. Alternatively, the processor 1202 can include another type of device, or multiple devices, configured for manipulating or processing information. For example, the processor 1202 can include multiple processors interconnected in one or more manners, including hardwired or networked. The operations of the processor 1202 can be distributed across multiple devices or units that can be coupled directly or across a local area or other suitable type of network. The processor 1202 can include a cache, or cache memory, for local storage of operating data or instructions.

The memory 1204 includes one or more memory components, which may each be volatile memory or non-volatile memory. For example, the volatile memory can be random access memory (RAM) (e.g., a DRAM module, such as DDR DRAM). In another example, the non-volatile memory of the memory 1204 can be a disk drive, a solid state drive, flash memory, or phase-change memory. In some implementations, the memory 1204 can be distributed across multiple devices. For example, the memory 1204 can include network-based memory or memory in multiple clients or servers performing the operations of those multiple devices.

The memory 1204 can include data for immediate access by the processor 1202. For example, the memory 1204 can include executable instructions 1216, application data 1218, and an operating system 1220. The executable instructions 1216 can include one or more application programs, which can be loaded or copied, in whole or in part, from non-volatile memory to volatile memory to be executed by the processor 1202. For example, the executable instructions 1216 can include instructions for performing some or all of the techniques of this disclosure. The application data 1218 can include user data, database data (e.g., database catalogs or dictionaries), or the like. In some implementations, the application data 1218 can include functional programs, such as a web browser, a web server, a database server, another program, or a combination thereof. The power source 1208 provides power to the computing device 1200. The peripherals 1210 includes one or more sensors, detectors, or other devices configured for monitoring the computing device 1200 or the environment around the computing device 1200. In some implementations, the computing device 1200 can omit the peripherals 1210.

The user interface 1212 includes one or more input interfaces and/or output interfaces. An input interface may, for example, be a positional input device, such as a mouse, touchpad, touchscreen, or the like; a keyboard; or another suitable human or machine interface device. An output interface may, for example, be a display, such as a liquid crystal display, a cathode-ray tube, a light emitting diode display, virtual reality display, or other suitable display.

The network interface 1214 provides a connection or link to a network. The network interface 1214 can be a wired network interface or a wireless network interface. The computing device 1200 can communicate with other devices via the network interface 1214 using one or more network protocols, such as using Ethernet, transmission control protocol (TCP), internet protocol (IP), another protocol, or a combination thereof.

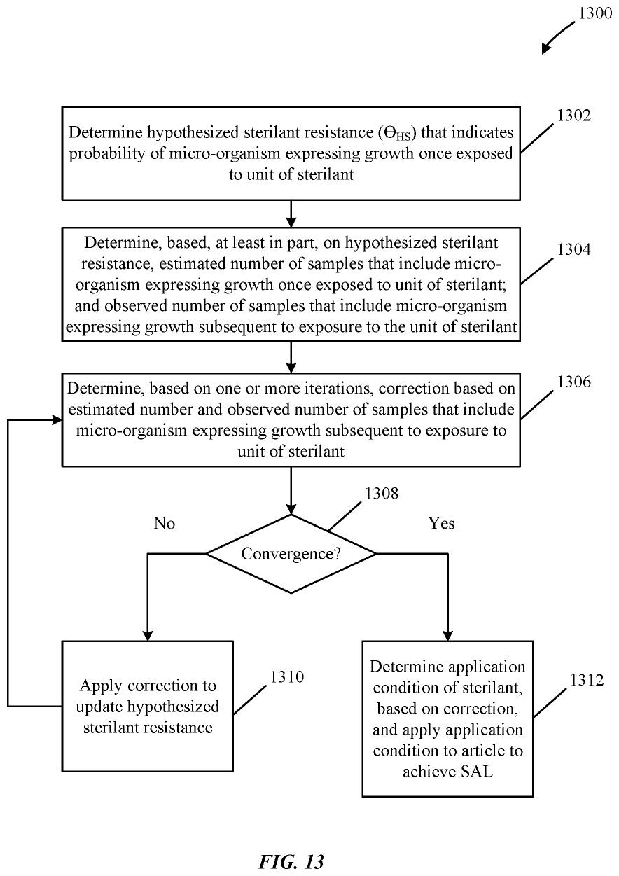

FIG. 13 is a flowchart of an example of a technique 1300 for determining a condition of sterilant to achieve an SAL for an article. The technique 1300 can be executed using computing devices, such as the systems, hardware, and software described with respect to FIGS. 1-12. The technique 1300 can be performed, for example, by executing a machine-readable program or other computer-executable instructions, such as routines, instructions, programs, or other code. The operations of the technique 1300 or another technique, method, process, or algorithm described in connection with the implementations disclosed herein can be implemented directly in hardware, firmware, software executed by hardware, circuitry, or a combination thereof.

For simplicity of explanation, the technique 1300 is depicted and described herein as a series of operations. However, the operations in accordance with this disclosure can occur in various orders and/or concurrently. Additionally, other operations not presented and described herein may be used. Furthermore, not all illustrated operations may be required to implement a technique in accordance with the disclosed subject matter.

At operation 1302, a system (e.g., the computing device 1200, implementing the EM algorithm 100 and/or the Bayesian MAP algorithm 200) may determine a hypothesized sterilant resistance (Θ_{HS}) that indicates a probability of a micro-organism expressing growth once exposed to a unit of sterilant. The unit of sterilant may correspond to dosage associated with a specific quantity, temperature, pressure, time, and/or another parameter. In some implementations, the sterilant may comprise radiation, Ethylene Oxide, dry heat, moist heat, or vaporized hydrogen peroxide. In some implementations, the operation 1302 may comprise the initialization operation of the EM algorithm 100. In some implementations, the operation 1302 may comprise the Bayesian MAP algorithm 200.

At operation 1304, the system may determine, based, at least in part, on the hypothesized sterilant resistance, an estimated number of samples that include a micro-organism expressing growth once exposed to the unit of sterilant. For example, an expectation function may generate an estimated number of samples that include a micro-organism expressing growth once exposed to the unit of sterilant. In some implementations, the operation 1304 may comprise the expectation operation of the EM algorithm 100 (e.g., the E-operation). In some implementations, the operation 1302 may comprise the prior operation of the Bayesian MAP algorithm 200 (e.g., a uniformed prior). The system may also determine an observed number of samples that include a micro-organism expressing growth subsequent to exposure to the unit of sterilant.

At operation 1306, the system may determine, based on one or more iterations, at least one correction based on the observed number and the estimated number. For example, the system may perform a maximization function following an expectation function. The maximization function may generate a correction based on the estimated number and an observed number of samples that include a micro-organism expressing growth once exposed to the unit of sterilant. In some implementations, including when the SAL is at least 10⁻⁶, the samples may be less than 100. The correction may represent a shift of an SAL curve that relates sterility assurance levels to units of sterilant (e.g., FIG. 6). In some implementations, the operation 1306 may be performed according to the EM algorithm 100. In some implementations, the operation 1306 may utilize a maximum log likelihood. In some implementations, determining the correction may include quantifying a positive difference between the estimated number and the observed number. In some implementations, determining the correction may include quantifying a negative difference between the estimated number and the observed number. In some implementations, the operation 1306 may comprise the maximization operation of the EM algorithm 100 (e.g., the M-operation). In some implementations, the operation 1306 may comprise the posterior operation of the Bayesian MAP algorithm 200.

At operation 1308, the system may apply convergence criteria to determine whether to perform a next iteration. For example, the system may receive input (e.g., user input through a graphical user interface) to define convergence criteria that identifies when a successful convergence is achieved (e.g., a point in FIG. 3 when applying the EM algorithm 100, or a point in FIG. 4 when applying the Bayesian MAP algorithm 200). If convergence criteria are not satisfied (“No”), the system may apply the correction (e.g., from operation 1306) to update the hypothesized sterilant resistance and return to operation 1306 to perform the next iteration. As a result, the system can repeat operation 1306 using an updated hypothesized sterilant resistance to determine a next correction. In some implementations, this may comprise a next iteration of the EM algorithm 100 (e.g., the E-operation and the M-operation). In some implementations, this may comprise a next iteration of the Bayesian MAP algorithm 200 (e.g., the prior-operation and the posterior-operation).

However, if convergence criteria are satisfied (“Yes”), at operation 1312 the system may determine an application condition of sterilant, based on the correction(s) that have been applied and based on the hypothesized sterilant resistance(s) that have been utilized and may apply the application condition to the article to achieve the SAL. For example, the system may reference an SAL curve (e.g., FIG. 6) that relates sterility assurance levels to units of sterilant to determine the condition. In some implementations, the condition may represent an amount, an absorbed dose, and/or a duration of exposure, including at a given temperature, and/or pressure, of the sterilant to achieve a minimum SAL given a microbial resistance of an actual article. While the disclosure has been described in connection with certain implementations, it is to be understood that the disclosure is not to be limited to the disclosed implementations but, on the contrary, is intended to cover various modifications and equivalent arrangements included within the scope of the appended claims, which scope is to be accorded the broadest interpretation so as to encompass all such modifications and equivalent structures as is permitted under the law.

What is claimed is:

1. A method for determining an application condition of sterilant to achieve a sterility assurance level (SAL) for an article, comprising:
a) determining a hypothesized sterilant resistance (Θ_{HS}) that indicates a probability of a micro-organism expressing growth once exposed to a unit of sterilant;
b) determining, based, at least in part, on the hypothesized sterilant resistance, an estimated number of samples that include a micro-organism expressing growth once exposed to the unit of sterilant;
c) determining an observed number of samples that include a micro-organism expressing growth subsequent to exposure to the unit of sterilant;
d) iteratively determining, based on one or more iterations, at least one first correction based on the observed number and the estimated number, and applying convergence criteria to determine whether to perform a next iteration; and
e) determining the application condition of the sterilant, based on the first correction, and applying the application condition to the article to achieve the SAL.

2. The method of claim 1, further comprising:
applying the first correction to update the hypothesized sterilant resistance; and
using the updated hypothesized sterilant resistance to determine a second correction, wherein determining the application condition is based on the second correction.

3. The method of claim 1, wherein d) is based on an expectation-maximization (EM) algorithm.

4. The method of claim 1, wherein d) utilizes a maximum log likelihood.

5. The method of claim 1, wherein determining the first correction includes quantifying a positive difference between the estimated number and the observed number.

6. The method of claim 1, wherein determining the first correction includes quantifying a negative difference between the estimated number and the observed number.

7. The method of claim 1, wherein the sterilant comprises at least one of radiation, Ethylene Oxide, dry heat, moist heat, or vaporized hydrogen peroxide.

8. The method of claim 1, wherein the application condition specifies an absorbed dose.

9. The method of claim 1, wherein the application condition specifies a duration of exposure at a given temperature or pressure.

10. The method of claim 1, wherein the first correction represents a shift of an SAL curve that relates sterility assurance levels to units of sterilant.

11. The method of claim 1, wherein the convergence criteria specifies a number of iterations.

12. The method of claim 1, further comprising:
referencing an SAL curve that relates sterility assurance levels to units of sterilant to determine the application condition.

13. The method of claim 1, wherein the SAL is at least 10⁻⁶ and the samples are less than 100 for achieving the SAL.

14. The method of claim 1, further comprising:
determining in b) an initialization selection that includes at least one of a resistance standard, a quantity or duration of exposure to the unit of sterilant, or number of samples.

15. The method of claim 1, further comprising:
determining an acceptance criterion comprising a fractional positive test of sterility (TOS).

16. The method of claim 1, wherein d) is based on a Bayesian maximum a posteriori (MAP) algorithm.

17. A non-transitory computer readable medium storing instructions operable to cause one or more processors to perform operations comprising:
determining a hypothesized sterilant resistance (Θ_{HS}) that indicates a probability of a micro-organism expressing growth once exposed to a unit of sterilant;
determining, based, at least in part, on the hypothesized sterilant resistance, an estimated number of samples that include a micro-organism expressing growth once exposed to the unit of sterilant;
determining an observed number of samples that include a micro-organism expressing growth subsequent to exposure to the unit of sterilant;
iteratively determining, based on one or more iterations, at least one first correction based on the estimated number and the observed number, and applying convergence criteria to determine whether to perform a next iteration; and
determining an application condition of sterilant, based on the first correction, and applying the application condition to the article to achieve a sterility assurance level (SAL).

18. The non-transitory computer readable medium storing instructions of claim 17, the operations further comprising:
applying the first correction to update the hypothesized sterilant resistance; and
using the updated hypothesized sterilant resistance to determine a second correction, wherein determining the application condition is based on the second correction.

19. The non-transitory computer readable medium storing instructions of claim 17, the operations further comprising:
applying, after determining the application condition, convergence criteria to determine whether to perform a next iteration.

20. The non-transitory computer readable medium storing instructions of claim 17, the operations further comprising:
referencing an SAL curve that relates sterility assurance levels to units of sterilant to determine the application condition.
